(19) United States
(12) Patent Application Publication
(10) Pub. No.: US 2008/0318233 A1
(43) Pub. Date: Dec. 25, 2008

(54) Source tagging and normalization of DNA for parallel DNA sequencing, and direct measurement of mutation rates using the same

(76) Inventors: Travis C. Glenn, Aiken, SC (US); Kenneth L. Jones, Winterville, GA (US); Stacey L. Lance, Aiken, SC (US); Gabor J. Szalai, Columbia, SC (US); Michael R. Felder, Columbia, SC (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 12/078,466

(22) Filed: Mar. 31, 2008

Related U.S. Application Data
(60) Provisional application No. 60/909,010, filed on Mar. 30, 2007.
(60) Provisional application No. 60/909,003, filed on Mar. 30, 2007.

(51) Int. Cl. C12Q 1/68

(52) U.S. Cl. 435/6

(57) ABSTRACT

This invention allows efficient tagging and normalization of DNA molecules prior to pooling and characterization using parallel DNA sequencing (e.g., commercially available 454 sequencers). The invention provides novel ways to process independent DNA samples (sources) so that similar numbers of molecules from each source are represented in the pool (i.e., the pool is normalized for representation among the sources). These approaches would save researchers time and energy relative to approaches currently available. In other embodiments, the invention provides novel ways to process large numbers of independently derived DNA samples that can be uniquely tagged in ways that allow the source of the DNA (e.g., an individual) to be tracked. The invention also provides novel ways for processing DNA to consistently obtain various portions of the genome to compare DNA sequences and directly measure mutations using state of the art massively parallel DNA sequencing (e.g., commercially available 454 sequencers and others. The invention is useful for the production of diagnostic assays for individuals prior to reproduction (e.g., cancer survivors who wish to procreate) and also for diagnostics of cancer (thus affecting the choice of treatment for cancer).

### CROSS REFERENCE TO RELATED APPLICATIONS

This application claims priority from U.S. provisional applications 60/909,010, filed Mar. 30, 2007, and 60/909,003, filed Mar. 30, 2007, both of which are incorporated herein by reference in their entirety.

### FIELD OF THE INVENTION

This invention relates generally to the field of nucleic acid analysis. More particularly, it concerns methods of tagging, normalization, and capture of DNA for use in massively parallel DNA sequencing. According to the methods of this invention, the source of the DNA can be efficiently tracked during parallel sequencing for such methods as genetic and genomic comparisons, mutation rate analysis, and assessment of DNA repair status.

### BACKGROUND

The new generation of pyro sequencers (e.g., 454 GS20 & FLX) produce 20-100 Mb of data per run through massively parallel reactions (e.g., 400,000 simultaneous reactions). Roche and 454 have proposed various approaches to sequence a great number of DNA fragments from individual samples. A major feature of those approaches is the requirement that specific sequences be on each end of DNA fragments to be sequenced. The reads from each individual sequence are, however, generally short (70-150 bases on the GS20 and 200-300 bases on the FLX).

It would be desirable to use the sequencing throughput of such instruments for population genetic analyses. However, population genetics requires the linking of sequence information to specific individuals. Traditionally, sequencing 1 gene from 10,000 individuals or 100 genes from 100 individuals have both required 10,000 reactions in independent tubes (or wells of plates) so that the user can track the source of the genetic information.

Thus, methods need to be developed to distinguish the source of the DNA to allow samples to be pooled and analyzed via parallel sequencing. Some progress has been made for tagging the source of DNA samples in 454 sequencing reactions. For example, Binladen et al., (2007) reports adding specific bases to the 5′ end of primers used for PCR. By keeping track of which primers were used with which individual, all amplicons can be traced back to the source DNA. This has numerous problems, including the need for large numbers of oligos for PCR and wasting a huge percentage of the sequenced bases (20-50%).

What is needed is an approach that: 1) minimizes the amount of sequence used for tracking purposes, 2) can work on multiple products from the same individual simultaneously, and 3) can work without modification to the original PCR primers. The current invention addresses the aforementioned problems and provides a solution to allow for direct measurement of mutation rates in germline and somatic cells using a combination of DNA tagging and/or selective hybridization and massively parallel DNA sequencing.

### BRIEF SUMMARY

In one embodiment, the invention provides methods to capture and tag one or more DNA fragments from one or more subjects to investigate one or more loci of interest. In one aspect, the method of the invention can be carried out in the following steps: (a) normalizing the concentration of the one or more DNA fragments, (b) pooling the one or more DNA fragments, (c) ligating distinct identification linker tags to each of the DNA fragments, (d) optionally pooling the distinctly tagged DNA fragments, (e) processing the distinctly tagged DNA fragments through parallel sequencing, and (f) using the identification linker tag to differentiate the one or more DNA fragments to investigate one or more loci of interest.

In another aspect, the invention provides a method to efficiently tag one or more DNA fragments and to use the tags to normalize the DNA fragments. The steps of this aspect of the invention comprise: (a) ligating a predefined amount of an identification linker tag to one or more DNA fragments, (b) pooling the one or more tagged DNA fragments, (c) repeating steps (a)-(b) for each subject, (d) optionally pooling the one or more tagged DNA fragments for all subjects, (e) capturing the one or more tagged DNA fragments, (f) purifying the one or more tagged DNA fragments, (g) releasing and reconstituting the one or more tagged DNA fragments, (h) processing the one or more tagged DNA fragments through parallel sequencing, and (i) using the identification linker tag to differentiate the one or more tagged DNA fragments from one or more subjects.

In another aspect, the invention provides a method for the measurement of mutations in the genome of a cell population by using tagged oligonucleotides to capture regions of interest prior to sequencing. The steps of this aspect of the invention comprise: (a) ligating a universal linker to one or more DNA fragments, (b) denaturing the one or more DNA fragments, (c) hybridizing the one or more DNA fragments with a tagged oligonucleotide, wherein the tagged oligonucleotide is complementary to the region of interest, (d) capturing the tagged oligonucleotide, (e) recovering the one or more DNA fragments containing the region of interest, (f) making the one or more DNA fragments double-stranded, (g) optionally removing the universal linker, and (h) processing the one or more DNA fragments through parallel sequencing to aid in the direct measurement of mutations in the genome of the cell population.

In another aspect, the invention provides a method of comparing portions of the genomes of one or more cells using tagged oligonucleotides to capture regions of interest prior to sequencing. The steps of this aspect of the invention comprise: (a) ligating a universal linker to one or more DNA fragments, (b) denaturing the one or more DNA fragments, (c) hybridizing the one or more DNA fragments with a tagged oligonucleotide, wherein the tagged oligonucleotide is complementary to the region of interest, (d) capturing the tagged oligonucleotide, (e) recovering the one or more DNA fragments containing the region of interest, (f) making the one or more DNA fragments double-stranded, (g) optionally removing the universal linker, and (h) processing the one or more DNA fragments through parallel sequencing to aid in comparing the genomes of one or more cells.

In another aspect, the invention provides a method for diagnosing cancer and other diseases involving altered DNA repair using tagged oligonucleotides to capture regions of interest prior to sequencing. The steps of this aspect of the invention comprise: (a) ligating a universal linker to one or more DNA fragments, (b) denaturing the one or more DNA fragments, (c) hybridizing the one or more DNA fragments with a tagged oligonucleotide, wherein the tagged oligonucleotide is complementary to the region of interest, (d) capturing the tagged oligonucleotide, (e) recovering the one or more DNA fragments containing the region of interest, (f) making the one or more DNA fragments double-stranded, (g) optionally removing the universal linker, (h) processing the one or more DNA fragments through parallel sequencing, and (i) comparing the genomes of one or more cells to help diagnose cancer and other diseases.

In another aspect, the invention provides a method for determining choice of treatment in a patient previously diagnosed with cancer and other diseases involving altered DNA repair using tagged oligonucleotides to capture regions of interest prior to sequencing. The steps of this aspect of the invention comprise: (a) ligating a universal linker to one or more DNA fragments, (b) denaturing the one or more DNA fragments, (c) hybridizing the one or more DNA fragments with a tagged oligonucleotide, wherein the tagged oligonucleotide is complementary to the region of interest, (d) capturing the tagged oligonucleotide, (e) recovering the one or more DNA fragments containing the region of interest, (f) making the one or more DNA fragments double-stranded, (g) optionally removing the universal linker, and (h) processing the one or more DNA fragments through parallel sequencing to aid in determining choice of treatment in a patient previously diagnosed with cancer and other diseases involving altered DNA repair.

In another aspect, the invention provides a method for the measurement of mutations in the genome of a cell population using oligonucleotides attached to a solid surface to capture regions of interest prior to sequencing. The steps of this aspect of the invention comprise: (a) ligating a universal linker to one or more DNA fragments, (b) denaturing the one or more DNA fragments, (c) hybridizing the one or more DNA fragments to one or more oligonucleotides complementary to the regions of interest and attached to a solid surface, (d) washing away unhybridized fragments, (e) recovering the one or more DNA fragments containing the region of interest, (f) making the one or more DNA fragments double-stranded, and (g) processing the one or more DNA fragments through parallel sequencing to aid in the direct measurement of mutations in the genome of the cell population.

In another aspect, the invention provides a method of comparing portions of the genomes of one or more cells using oligonucleotides attached to a solid surface to capture regions of interest prior to sequencing. The steps of this aspect of the invention comprise: (a) ligating a universal linker to one or more DNA fragments, (b) denaturing the one or more DNA fragments, (c) hybridizing the one or more DNA fragments to one or more oligonucleotides complementary to the regions of interest and attached to a solid surface, (d) washing away unhybridized fragments, (e) recovering the one or more DNA fragments containing the region of interest, (f) making the one or more DNA fragments double-stranded, and (g) processing the one or more DNA fragments through parallel sequencing to aid in the direct measurement of mutations in the genome of the cell population.

In another aspect, the invention provides a method for diagnosing cancer and other diseases involving altered DNA repair using oligonucleotides attached to a solid surface to capture regions of interest prior to sequencing. The steps of this aspect of the invention comprise: (a) ligating a universal linker to one or more DNA fragments, (b) denaturing the one or more DNA fragments, (c) hybridizing the one or more DNA fragments to one or more oligonucleotides complementary to the regions of interest and attached to a solid surface, (d) washing away unhybridized fragments, (e) recovering the one or more DNA fragments containing the region of interest, (f) making the one or more DNA fragments double-stranded, and (g) processing the one or more DNA fragments through parallel sequencing to aid in the direct measurement of mutations in the genome of the cell population.

In another aspect, the invention provides a method for determining choice of treatment in a patient previously diagnosed with cancer and other diseases involving altered DNA repair using oligonucleotides attached to a solid surface to capture regions of interest prior to sequencing. The steps of this aspect of the invention comprise: (a) ligating a universal linker to one or more DNA fragments, (b) denaturing the one or more DNA fragments, (c) hybridizing the one or more DNA fragments to one or more oligonucleotides complementary to the regions of interest and attached to a solid surface, (d) washing away unhybridized fragments, (e) recovering the one or more DNA fragments containing the region of interest, (f) making the one or more DNA fragments double-stranded, and (g) processing the one or more DNA fragments through parallel sequencing to aid in the direct measurement of mutations in the genome of the cell population.

### BRIEF DESCRIPTION OF THE DRAWINGS/FIGURES

FIG. 1 compares the traditional and new normalization approaches.

FIG. 2 is a schematic showing the process of tagging and normalizing source DNA for parallel sequencing.

FIG. 3 represents variations of the linker tagging process.

FIG. 4 is a gel electrophoresis image of PCR amplifications of Mus DNA enriched using capture techniques appropriate for massively parallel DNA sequencing to compare genomes, mutation rates, and/or DNA repair efficiency.

FIG. 5 is a graph showing normalization of three different PCR products following bead capture and elution.

FIG. 6 shows results of qPCR following normalization of PCR products initially varying by 4 orders of magnitude.

### DETAILED DESCRIPTION

### Definitions and Materials

The invention involves appending a “tag” to one or more target sequences. As used herein, a tag is a common sequence shared by various nucleic acid sequences of a sample that allows nucleic acids of one sample to be distinguished from nucleic acids from another sample. In one embodiment, the tag could be a nucleic acid sequence. The tag could be DNA, or derivative or analog thereof. The tag could also be RNA, or derivative or analog thereof. The tag could also be used as a template by a polymerase to generate a complementary strand.

A tag may be made by any technique known to one of ordinary skill, such as chemical synthesis. Non-limiting examples of methods of chemical synthesis of a tag include generation of synthetic nucleic acids using phosphotriester, phosphite or phosphoramidite chemistry and solid phase techniques. The tag may also be made by enzymatic production. A non-limiting example of enzymatically produced nucleic acids includes one produced by enzymes in amplification reactions, such as PCR or the synthesis of an oligonucleotide. The tag may also be produced by biological production. A non-limiting example of a biologically produced nucleic acid includes recombinant nucleic acids produced in a living cell, such as a DNA vector replicated in bacteria.

A nucleic acid tag of the present invention may be added to or appended to a nucleic acid population. In one embodiment, the tag can be appended to the nucleic acid population by a cloning process. The tag could be appended to the nucleic acid population by ligation. The tag could also be appended to the nucleic acid population by blunt-end ligation. The tag could also be appended to the nucleic acid population by ligation to a 5′ or 3′ overhang in the DNA sequence. As would be appreciated by one of ordinary skill, different methods of tag attachment or incorporation may be used.

In one embodiment, a tag or nucleic acid used in the present invention could be purified on a gel. A gel could include polyacrylamide gels. A gel could also include cesium chloride centrifugation gradients, or any other means known to one of ordinary skill.

As used herein, “sources” can include, but are not limited to, humans or other animals, plants, bacteria, or viruses. In other embodiments, a source can refer to tissues, cells, tumor tissues, tumor cells, plant cells, or any other biological material from which DNA can be extracted. In one embodiment, the DNA fragments used in the invention come from different sources. In another embodiment, the different sources are various tissue types (e.g., normal or cancerous epithelium, connective, muscle, or nervous etc.). In another embodiment, the different sources are various subjects. Reference herein of “a subject” or “all subjects” can include a single subject or multiple subjects. Reference herein of “pooling” DNA fragments of all subjects can include pools of one subject, pools of all subjects, or pools of any combination of more than one subject. A subject can include eukaryotes or prokaryotes. This includes humans or other animals, plants, bacteria, or viruses. In one embodiment, the distinctly tagged DNA fragments used in the claimed invention are pooled from a plurality of different sources. In another embodiment, each source has a distinct identification linker tag.

For sake of convenience, the present specification refers throughout to DNA. It is understood, however, that the methods of the present invention can also be used to tag and analyze RNA, including mRNA, tRNA, rRNA, and snRNA.

As used herein, “DNA fragments” can include, but are not limited to, polymerase chain reaction (PCR) amplicons, DNA generated through rolling circle amplification (RCA) or cDNA synthesis, or DNA fragmented by physical means or restriction enzyme digestion. The DNA could be isolated from at least one organelle, cell, tissue or organism. The organism could be a prokaryote or eukaryote. The DNA could come from genomic DNA. The genomic DNA could come from somatic cells or germline cells. Additionally, the genomic DNA could be isolated from tumor cells. The DNA could also come from plasmid, cosmid, fosmid, or BAC DNA. The DNA could also come from bacterial DNA. The DNA could also come from an animal or plant source. In another embodiment, the DNA fragment(s) is/are isolated from mitochondrial DNA.

In one embodiment, the DNA obtained from a source can be crudely liberated. Crude liberation of DNA would include, for example, collection of DNA from a cell lysis without any further processing to purify the DNA. In another embodiment, the DNA obtained from a source would be purified such that no, or very few, proteins, RNA, or lipids are contained in the solution. In another embodiment, the DNA is obtained by whole genome amplification. Whole genome amplification could include amplification by PCR or any other amplification process yielding strands of DNA. In other embodiments, the source DNA can be relatively to highly purified.

As used herein, an “amplicon” means the product of an amplification reaction. That is, it is a population of polynucleotides that are replicated from one or more starting sequences. In one embodiment, the polynucleotides could be single stranded. The polynucleotides could also be double stranded. The one or more starting sequences may be one or more copies of the same sequence, or it may be a mixture of different sequences. In one embodiment, the amplicons may be produced in a PCR reaction. The amplicons could also be produced by replication in a cloning vector. The amplicons could also be produced by linear amplification by an RNA polymerase, such as T7 or SP6, or by any like techniques.

As used herein, “locus”, or the plural form “loci”, in reference to a genome or target polynucleotide(s), means a contiguous subregion(s) or segment(s) of the genome or target polynucleotide. In one embodiment, locus, or loci, may refer to the position(s) of a gene or genes in a genome. A locus, or loci, could also refer to the position(s) of a portion of a gene or genes in a genome. In another embodiment, a locus, or loci, may refer to any contiguous portion of genomic sequence within, or associated with, a gene. A locus, or loci, could also refer to any contiguous portion of a genomic sequence not within, or not associated with, a gene. In another embodiment, a locus, or loci, could refer to an exonic region of DNA. A locus, or loci, could also refer to an intronic region of DNA.

As used herein, “ligating” (or to “ligate” or “ligation”) means to form a covalent bond or linkage between the termini of two or more nucleic acids, e.g. oligonucleotides and/or polynucleotides, in a template-driven reaction. The nature of the bond or linkage may vary widely. In one embodiment, the ligation may be carried out enzymatically. In another embodiment, the ligation may be carried out chemically. The ligation may occur through the action of a ligase. DNA ligase is a type of ligase that can link together DNA strands that have double-strand breaks (a break in both complementary strands of DNA). The ligase could be an enzymatic protein. The ligase could also be a nucleic acid that induces ligation. Also, the ligase could be a chemical that induces ligation. In one embodiment, ligation is performed to generate a tag on a DNA fragment.

As used herein, a “linker” or “universal linker” refers to a double-stranded oligonucleotide that carries particular sequences useful in carrying out the present invention. The terms “linker” and “universal linker” can be used interchangeably in this invention. In one embodiment, the linker contains an identifying sequence. This sequence is a unique sequence for the respective linker. In one embodiment, the linker contains a unique sequence of nucleotides. This unique sequence of nucleotides serves as a “tag,” as previously defined. This unique sequence of nucleotides together with a linker can be referred to interchangeably as a “linker tag” or a “unique linker tag” or an “ID linker tag” or a “unique ID linker” or a “unique ID linker tag”. The unique linker tag contains an identifying sequence of 1 or more nucleotides that serve as a means to tag a desired DNA fragment once the linker is ligated on to the DNA fragment. The unique sequence of nucleotides can be observed following sequencing such that one could distinguish the source of the DNA fragment.

In one embodiment, the identification linker tag is a 1 mer to about a 100 mer. In some embodiments, the linker tag is about 10 to about 30 bases, or about 10 to about 20 bases. In at least some embodiments, the linker could be a 1 mer. In another embodiment, the linker is about a 100 mer. In certain embodiments, the linker will contain multiple unique nucleotides relative all other linkers to help the user identify the linker sequence.

In one embodiment, the linker or universal linker can be SuperSNX (SEQ ID NOS:23-24). In another embodiment, the linker or universal linker can be 454 As explained elsewhere in the present specification, the linker provides a known sequence for purposes of tagging the DNA fragment of interest. Therefore, how to make such linkers are well within the purview of the person of ordinary skill in the art.

In one embodiment, the linker can contain a “key sequence”. A key sequence is a unique sequence of nucleotides that is recognized by the high throughput sequencers used in the various embodiments of the invention. The key sequence is variable, depending on the sequencing platform used. These key sequences are known to those of skill in the art.

In one embodiment, the linker can be ligated to an amplicon. The amplicon can be linked to the linker on only one end of the amplicon. The amplicon can also have a linker on both ends. In one embodiment, the amplicon can have the same linker on both ends. In another embodiment, the amplicon can have different linkers on each end. In one embodiment, the identification linker tags are ligated individually. In another embodiment, they are ligated simultaneously. In another embodiment, the identification linker tags are ligated in multiple steps. This steps can include restriction enzyme digestion and/or cloning steps.

In one embodiment, the linker can be ligated to a DNA fragment. The DNA can be blunt-ended or have single or multiple base overhangs. The DNA fragment can be linked to the linker on only one end of the fragment. The DNA fragment can also have a linker on both ends. In one embodiment, the DNA fragment can have the same linker on both ends. In another embodiment, the DNA fragment can have different linkers on each end. In one embodiment, the identification linker tags are ligated individually. In another embodiment, they are ligated simultaneously. In another embodiment, the identification linker tags are ligated in multiple steps. These steps can include restriction enzyme digestion and/or cloning steps. Ligation protocols and variations on ligation reactions are all well known to those skilled in the art.

In one embodiment, the linker can also be linked, or attached, to a labeling moiety The labeling moiety could be biotin. Biotin is a molecule often chemically linked, or tagged, to a molecule (such as DNA or RNA or protein) for biochemical assays. This process of linking biotin is called biotinylation. Avidin is a glycoprotein that has a very strong affinity for biotin. Since avidin binds preferentially to biotin, biotin-tagged molecules can be extracted from a sample by mixing them with beads with covalently-attached avidin, and washing away anything unbound to the beads.

The labeling moiety could also be TOPOisomerase. Also, the moiety attached to the linker could be digoxigenin. Digoxigenin is a non-radioactive DNA label used in a wide range of chemical and biological applications, including, for example, Southern blotting, Dot blotting, arrays, colony hybridization, in situ hybridization, and in enzyme-linked immunosorbent assays (ELISA). Also, the moiety attached to the linker could be fluorescein isothiocyanate (FITC), or any other such immunological reagents. FITC is a derivative of fluorescein used in wide-ranging chemical and biological applications where fluorescent labeling is desirable. Examples of applications for FITC include flow cytometry and as immunohistochemical markers in in situ hybridization. In one embodiment, the linker is phosphorylated on one end. The linker could also be phosphorylated on both ends.

In one embodiment, one or more additional linkers are ligated to the DNA fragment(s) prior to parallel sequencing. In another embodiment, the additional linkers contain unique DNA sequences. These unique DNA sequences can include the identification sequence with additional base pairs 3′ or 5′

In one embodiment, labile biotin can be used to capture the identification linker tagged DNA fragment(s). Labile biotin can be reversibly bound to avidin/streptavidin and thus provides another avenue in which captured fragments can be released for further processing. As would be understood by those skilled in the art, other similar methods for capture could be used interchangeably with this invention and result in minor modifications to the described process.

In one embodiment, the linker and/or DNA molecules of interest can be captured using oligonucleotides attached to solid surfaces including but not limited to glass slides, nylon membranes, nitrocellulose membranes, paramagnetic particles, magnetic particles, and particles with a density different from water. Following hybridization and removal of unbound DNA through washing, DNA bound to the solid surface can be removed and used as template for sequencing (Albert et al. 2007).

As used herein, “parallel sequencing” refers to sequencing using massively parallel DNA sequencing (e.g., commercially available high speed, high throughput pyro sequencers, such as the 454 GS20 and FLX sequencers (Roche and 454 Life Sciences) and others. As would be understood by those skilled in the art, other similar sequencing methods and/or platforms could be used interchangeably with this invention.

As used herein, “sequencing” or “DNA sequencing” refers to biochemical methods for determining the order of the nucleotide bases, adenine, guanine, cytosine, and thymine, in a DNA oligonucleotide. Sequencing, as the term is used herein, can include parallel sequencing or any other sequencing method known of those skilled in the art, for example, chain-termination methods, rapid DNA sequencing methods, wandering-spot analysis, Maxam-Gilbert sequencing, dye-terminator sequencing, or using any other modern automated DNA sequencing instruments.

“Patient” as used herein means human or non-human patients. Examples of non-human patients include, but are not limited to, scientifically useful test animals (such as mice and rats), commercially important animals (such as cows, sheep, and pigs), as well as common pet animals (such as dogs and cats).

### Methods and Uses of the Invention

1. Method for Tagging

In one aspect of the present invention, a method is provided to tag one or more DNA fragments from one or more subjects to investigate one or more loci of interest comprising: (a) normalizing the concentration of the one or more DNA fragments, (b) pooling the one or more DNA fragments, (c) ligating distinct identification linker tags to each of the DNA fragments, (d) optionally pooling the distinctly tagged DNA fragments, (e) processing the distinctly tagged DNA fragments through parallel sequencing, and (f) using the identification linker tag to differentiate the one or more DNA fragments to investigate one or more loci of interest.

Normalization can be done by quantitation and dilution of the DNA fragments. It is understood that by normalization as used herein, generally accepted procedures of DNA quantitation by one of skill in the art are used. This can include DNA quantitation based on spectrophotometric (OD) readings, gel electrophoresis, fluorometric quantification methods, or any other accepted method of DNA quantitation. It is also understood that dilution of DNA is done by generally accepted procedures of diluting DNA. This can include diluting a DNA solution in water or any other acceptable solution, such as buffered solutions. The purpose of normalization is to start with approximately equal amounts of starting material (i.e. DNA) during experimentation such that accurate quantitation of enrichment is possible and sample results can be interpreted consistently. Generally, normalization yields approximately equal molar amounts of the starting material. In other embodiments, the normalization yields equal molar amounts of the starting material.

Pooling of DNA fragments is the combining of the source(s) DNA into a single workable unit, or aliquot. Pooling of DNA fragments can be done by collecting all of the DNA fragments into a single aliquot, or multiple aliquots if necessary. The DNA can be pooled in any suitable solution where ligation would not be hindered. In one embodiment, tens or hundreds of DNA fragments are pooled into a single aliquot. In other embodiments, hundreds of thousands to millions of separate DNA fragments are pooled into a single aliquot. In other embodiments, up to millions of separate DNA fragments are pooled into a plurality of aliquots, generally between 2 and 96 separate aliquots.

Distinct identification linker tags can be ligated to each of the DNA fragments. Ligation of distinct identification linker tags can be performed using an appropriate enzymatic or chemical ligation, as previously embodiments explain. Suitable enzymes for ligation are well known to those skilled in the art. For example, DNA ligase I, II, III, or IV could be suitable ligases.

Pooling can be performed for the distinctly tagged DNA fragments. The fragments can be combined into a single workable unit, or aliquot. The DNA can be pooled in any suitable solution where sequencing would not be hindered. The pool can be generated to provide for a single pool to sample from when doing subsequent sequencing.

Sequencing of the DNA fragments can be done. As defined previously, parallel sequencing can be used. As would be understood by those skilled in the art, other similar sequencing methods and/or platforms could be used interchangeably with this invention.

The identification linker tag(s) can be used to differentiate the one or more DNA fragments to investigate one or more loci of interest. The tagged DNA fragment sequence can be analyzed by any method used in the art. In one embodiment, such methods include computerized algorithms to analyze sequence information. Differentiation of DNA fragment sources can be determined by the unique sequence located on the linker tag.

In addition to patient DNA, as that term was previously defined, it is understood that the methods of the present invention are applicable to the study of microbial organisms, such as bacteria, yeast, fungi, protozoa, and the like. The present invention can also be used in the study of viruses, including HIV, HPV, herpes virus, and the influenza virus.

2. Method for Efficiently Tagging and/or Normalizing DNA Fragments

In another aspect of the invention, a method is provided to efficiently tag and/or normalize one or more DNA fragments comprising: (a) ligating a predefined amount of an identification linker tag to one or more DNA fragments, (b) pooling the one or more tagged DNA fragments, (c) repeating steps (a)-(b) for each subject, (d) optionally pooling the one or more tagged DNA fragments for all subjects, (e) capturing the one or more tagged DNA fragments, (f) purifying the one or more tagged DNA fragments, (g) releasing and reconstituting the one or more tagged DNA fragments, (h) processing the one or more tagged DNA fragments through parallel sequencing, and (i) using the identification linker tag to differentiate the one or more tagged DNA fragments from one or more subjects.

A predefined amount of an identification linker tag can be ligated to the DNA fragment(s) to be analyzed. In this embodiment, a limited amount of the linker could be ligated to the DNA fragments. In this embodiment, the amount, or concentration, of DNA fragments reacted with the linker is not limiting. The amount of DNA used in the reaction with the linker could be in any amount, or concentration, in excess of the amount of linker used in the reaction. When the concentration is properly adjusted, you get normalized amounts of tagged molecules over a very broad range of DNA fragment concentrations from PCR analysis. Empirically, this can be a trial and error process where one could observe the results and adjust the amounts of linker accordingly (amounts of linker that are too low or too high will not normalize). This is well within the ordinary skill level of those trained in the art.

Pooling can be performed for the distinctly tagged DNA fragments. The fragments can be combined into a single workable unit, or aliquot. The DNA can be pooled in any suitable solution where sequencing would not be hindered. The pool can be generated to provide for a single pool to sample from when doing subsequent sequencing.

Ligation of the linker to the DNA fragments can be repeated for any number of DNA sources. The number of sources is not limited. Each source can be pooled within its respective source pool and then among any number of sources. A single pool can be obtained containing some, or all, DNA fragments from any number of sources combined. A single pool can also be obtained containing some, or all, DNA fragments from only a single source. Each source can have a unique identification tag within the linker. Each source can also have several unique identification tags within the linker. Also, the same identification tag can be used for multiple sources. The distinctly tagged DNA fragments used in the claimed method can be pooled from a plurality of different sources.

In one embodiment, the one or more tagged DNA fragments are not pooled. In another embodiment, both ends of the one or more DNA fragments are ligated to linkers lacking unique identification sequences. In another embodiment, the one or more tagged DNA fragments are not pooled and both ends of the one or more DNA fragments are ligated to linkers lacking unique identification sequences

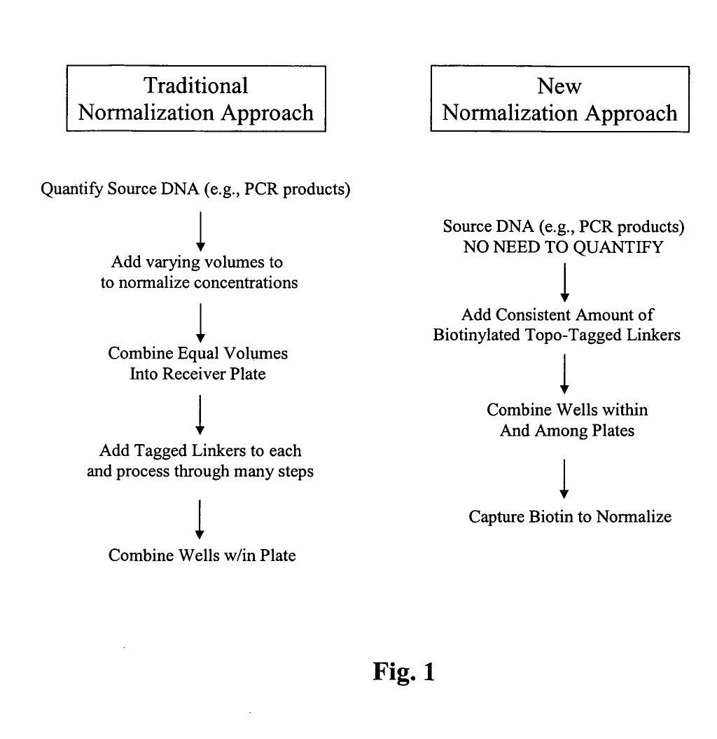

Capturing the tagged DNA fragments can be performed by any method previously described herein. For example, biotin could be used. In one embodiment, labile biotin can be used to capture the identification linker tagged DNA fragment(s). Digoxigenin could be used to capture the tagged DNA fragments. Also, FITC or any other suitable immunological reagent known to those skilled in the art, could be used to capture the tagged DNA fragments. Efficient normalization of DNA fragments is obtained by this process, as characterized in FIG. 1.

Purification of the tagged DNA fragments can be performed. Purification can be used to remove any DNA that is present without the appropriate tag or labeling moiety. Purification can also be used to remove other reagents, unligated linkers, nucleotides, enzymes, or other impurities from the reaction. Purification can be accomplished by any acceptable means used by those skilled in the art. Examples of purification techniques include, but are not limited to, centrifugation, enzyme treatments, gel electrophoresis, or DNA precipitation using salt solutions or any other acceptable DNA precipitation buffer.

Releasing and reconstituting the DNA fragments can be performed. Release of the DNA fragments from the capturing moiety previously described could be accomplished by any acceptable procedure used in the art. Examples of possible release procedures include, but are not limited to, heat treatment, chemical treatment, or enzymatic treatment. The DNA fragments can be reconstituted by any acceptable method known in the art. Reconstitution of DNA could be in water. The DNA could also be reconstituted in a saline solution or any other buffered solution used in the art.

Sequencing of the DNA fragments can be done. As defined previously, parallel sequencing or any other previously described sequencing procedure could be used.

The identification linker tag(s) can be used to differentiate the one or more tagged DNA fragments from one or more subjects The tagged DNA fragment sequence can be analyzed by any method used in the art or described previously herein. Differentiation of DNA fragment sources can be determined by the unique sequence located on the linker tag, allowing for the distinction of DNA from each subject.

3. Method for Measurement of Mutations in a Genome

In another aspect of the invention, a method is provided for the measurement of mutations in the genome of a cell population by identifying and sequencing a region of interest comprising: (a) ligating a universal linker to one or more DNA fragments, (b) denaturing the one or more DNA fragments, (c) hybridizing the one or more DNA fragments with a tagged oligonucleotide, wherein the tagged oligonucleotide is complementary to the region of interest, (d) capturing the tagged oligonucleotide, (e) recovering the one or more DNA fragments containing the region of interest, (f) making the one or more DNA fragments double-stranded, (g) optionally removing the universal linker, and (h) processing the one or more DNA fragments through parallel sequencing to aid in the direct measurement of mutations in the genome of the cell population. In one embodiment, steps (c)-(f) of the method are repeated before proceeding to step (g).

A universal linker can be ligated to the DNA fragments. In one embodiment, the universal linker may contain an identification tag. In some embodiments, the universal linker may not be removed during the method of the invention.

The DNA fragments can also be denatured. As used herein, “denaturing” refers to the process by which double-stranded deoxyribonucleic acid unwinds and separates into single-stranded strands through the breaking of hydrogen bonding between the bases. Denaturation of DNA can be accomplished by any method used by those of skill in the art. DNA can be denatured by, for example, heating the DNA or contacting the DNA with chemicals. Chemicals involved in denaturing DNA are well known in the art, and can include urea.

“Complementary” as used herein in reference to tagged oligonucleotides refers to a tagged oligonucleotide to which an oligonucleotide or other DNA sequence specifically hybridizes to form a perfectly matched duplex.

As used herein, “hybridizing” refers to the forming of a double or triple stranded molecule or a molecule with partial or double or triple stranded nature. It is understood by those of skill in the art that hybridization is accomplished either under “stringent”, “low stringency”, or “high stringency” conditions. High stringency conditions are those conditions that allow for hybridization between one or more nucleic acid strands containing complementary sequences, but precludes hybridization of random sequences. Stringent or low stringency conditions are those conditions that allow for hybridization between one or more nucleic acid strands that could not hybridize under “high stringency” conditions. Such hybridized sequences may not be entirely complementary strands, and may contain some random sequences.

As used herein, the “region of interest” is any DNA sequence or fragment of DNA in which one wishes to form a complementary hybridization with an oligonucleotide.

The DNA fragments can be hybridized with a tagged oligonucleotide. This hybridization process could be repeated for the same source, or multiple sources. The tagged oligonucleotide can also be complementary to the region of interest. Depending on the application, stringent, low stringency, or high stringency conditions could be used for the hybridization.

Capturing the tagged oligonucleotide can be performed by any method previously described herein or known by those of skill in the art. For example, the tagged oligonucleotide could be linked to any of the previously described labeling moieties, biotin, digoxigenin, or FITC could be used. This capturing process could be repeated for the same source, or multiple sources. Additionally, capturing the tagged oligonucleotide and/or region of interest can be performed by hybridization to oligonucleotides attached to solid support including but not limited to glass slides, nylon membranes, nitrocellulose membranes, paramagnetic particles, magnetic particles, and particles with a density different from water.

DNA fragments containing the region of interest can be recovered by methods recognized by those skilled in the art. For example, if biotin is linked to the tagged oligonucleotides, avidin, as previously described, could be used to recover the DNA fragments containing the region of interest. Similarly, DNA fragments hybridized to solid surfaces can be recovered using high stringency washes (i.e., stripping). This recovery process could be repeated for the same source, or multiple sources.

The DNA fragments can also be made double-stranded. This process could be repeated for the same source, or multiple sources. The introduction of the universal linker can facilitate the process of making the DNA fragment double stranded. Additionally, those skilled in the art would recognize methods of making DNA double stranded through the use of appropriate polymerases and other enzymes.

In one embodiments, the universal linker can be removed. In another embodiment, the universal linker is not removed. Removal of the universal linker would be dependent on the application. For example, if one wished to determine the source of the DNA fragment, a universal linker containing a unique identification tag could be retained for identification of the source of the DNA fragment.

Sequencing of the DNA fragments can be done. As defined previously, parallel sequencing or any other previously described sequencing procedure could be used.

The sequence obtained could aid in the direct measurement of mutations in the genome of the cell population. In one embodiment, the region of interest contains a region (or regions) with at least one microsatellite repeat. Microsatellites are well known to have mutation rates higher than the average single-copy nuclear DNA and also higher relative to the rest of the genome. The region of interest could also be within another intronic region. The region of interest could also be within an exonic region.

In one embodiment, a “peptide nucleic acid” or tagged peptide nucleic acid can be substituted for the tagged oligonucleotide. As used herein, a peptide nucleic acid, also known as a peptide-based nucleic acid analog, generally comprises one or more nucleotides or nucleosides that comprise a nucleobase moiety, a nucleobase linker moiety that is not a 5-carbon sugar, and/or a backbone moiety that is not a phosphate backbone moiety. The peptide nucleic acid could contain a unique identification tag.

4. Method for Comparing Genomes

One embodiment of the present invention allows for the direct estimate of germline mutation rates in animals from many thousands or millions of offspring, and/or mutation accumulation lines many generations old. The use of highly mutable loci (such as ESTRs or STRs) also allows for estimates based on much smaller numbers of individuals.

There is, however, an alternative strategy that makes use of the new generation of pyro sequencers (e.g., 454 FLX)-producing ˜100 Mb of data per run through massively parallel reactions. Collecting such a large amount of data per run would allow assessment of portions of the genome with much lower mutation rates than ESTRs or STRs and would allow a huge number of loci to be assessed simultaneously. However, there are three key problems. The first problem is that much of the genome mutates at a rate that is too low to be detected (e.g., if the mutation rate is 1×10⁸ per base pair, assaying 1×10⁸ base pairs will probably only reveal one mutation). Second, these instruments are currently only able to assay a small percentage of a typical vertebrate genome (about 3% of the human genome or 20% of the medakafish genome). If one were to try to assay the whole genome, one would not get enough information from an individual to be able to readily compare it with information from another individual (i.e., the 3% of the genome assayed from individual A will only have very little in common with 3% of the genome assayed from individual B). And third, because the reads from each individual sequence are short (˜200 nucleotides), these cannot sequence across long perfect microsatellites (ESTRs and long STRs) which are the loci with the highest mutation rates. Thus, direct assessment of mutations in vertebrate genomes using currently known techniques cannot be done on these instruments at this point in time. Indeed, this and other factors lead to the infeasibility of sequencing whole vertebrate genomes on these instruments at this point in time.

The current invention addresses the aforementioned problems and provides a solution to allow for direct measurement of mutation rates in germline and somatic cells using a combination of DNA tagging and/or selective hybridization and massively parallel DNA sequencing.

In one aspect of the invention, a method is provided for comparing portions of the genomes of one or more cells comprising: (a) ligating a universal linker to one or more DNA fragments, (b) denaturing the one or more DNA fragments, (c) hybridizing the one or more DNA fragments with a tagged oligonucleotide, wherein the tagged oligonucleotide is complementary to the region of interest, (d) capturing the tagged oligonucleotide, (e) recovering the one or more DNA fragments containing the region of interest, (f) making the one or more DNA fragments double-stranded, (g) optionally removing the universal linker and (h) processing the one or more DNA fragments through parallel sequencing to aid in comparing the genomes of one or more cells. In one embodiment, steps (c)-(f) of the method are repeated before proceeding to step (g).

A universal linker can be ligated to the DNA fragments. In one embodiment, the universal linker may contain an identification tag. In some embodiments, the universal linker may not be removed during the method of the invention.

The DNA fragments can also be denatured.

The DNA fragments can be hybridized with a tagged oligonucleotide. This hybridization process could be repeated for the same source, or multiple sources. The tagged oligonucleotide can also be complementary to the region of interest. Depending on the application, stringent, low stringency, or high stringency conditions could be used for the hybridization.

Capturing the tagged oligonucleotide can be performed by any method previously described herein or known by those of skill in the art. For example, the tagged oligonucleotide could be linked to any of the previously described labeling moieties, biotin, digoxigenin, or FITC could be used. Additionally, capturing the tagged oligonucleotide and/or region of interest can be performed by hybridization to oligonucleotides attached to solid surface/support including but not limited to glass slides, nylon membranes, nitrocellulose membranes, paramagnetic particles, magnetic particles, particles with a density different from water. This capturing process could be repeated for the same source, or multiple sources.

The region of interest in the present invention can be identified from DNA sequences conserved among divergent taxa. The concept of divergent taxa would be understood by those skilled in the art. Also, the region of interest can occur once, or multiple, times within the genome of the organism under study. The oligonucleotides complementary to the region of interest can also include a repetitive element or flank repetitive elements.

DNA fragments containing the region of interest can be recovered by methods recognized by those skilled in the art. For example, if biotin is linked to the tagged oligonucleotides, avidin, as previously described, could be used to recover the DNA fragments containing the region of interest. Similarly, DNA fragments hybridized to microarrays can be recovered using high stringency washes (i.e., stripping). This recovery process could be repeated for the same source, or multiple sources.

The DNA fragments can also be made double-stranded. This process could be repeated for the same source, or multiple sources. The introduction of the universal linker can facilitate the process of making the DNA fragment double stranded. Additionally, those skilled in the art would recognize methods of making DNA double stranded through the use of appropriate polymerases and other enzymes.

In one embodiments, the universal linker can be removed. In another embodiment, the universal linker is not removed. Removal of the universal linker would be dependent on the application. For example, if one wished to determine the source of the DNA fragment, a universal linker containing a unique identification tag could be retained for identification of the source of the DNA fragment.

In one embodiment, the region of interest contains a region (or regions) with at least one microsatellite repeat. The region of interest could also be within another intronic region. The region of interest could also be within an exonic region.

In one embodiment, as previously defined, a peptide nucleic acid or tagged peptide nucleic acid can be substituted for the tagged oligonucleotide.

Sequencing of the DNA fragments can be done. As defined previously, parallel sequencing or any other previously described sequencing procedure could be used.

The sequence obtained could aid in comparing the genomes of one or more cells. In one embodiment, the universal linker could contain a unique identification tag. By not removing the tag, one could determine the source of the DNA based on the unique identification tag. A unique identification tag could be used for each cell. A unique identification tag could also be used for each group of cells. Each group of cells could be from a single source or multiple sources.

5. Method for Diagnosing Cancer and Other Diseases

In another aspect of the invention, a method is provided for diagnosing cancer and other diseases involving altered DNA repair comprising: (a) ligating a universal linker to one or more DNA fragments, (b) denaturing the one or more DNA fragments, (c) hybridizing the one or more DNA fragments with a tagged oligonucleotide, wherein the tagged oligonucleotide is complementary to the region of interest, (d) capturing the tagged oligonucleotide, (e) recovering the one or more DNA fragments containing the region of interest, (f) making the one or more DNA fragments double-stranded, (g) optionally removing the universal linker, (g) processing the one or more DNA fragments through parallel sequencing, and (h) comparing the genomes of one or more cells to help diagnose cancer and other diseases. In one embodiment, steps (c)-(f) of the method are repeated before proceeding to step (g).

It is understood that the methods of the present invention are applicable to the study of cancer and other diseases. Other diseases can include, but are not limited to, heart disease, genetic disorders, Alzheimer's disease, diabetes, Huntington's disease, or sickle cell anemia.

In this embodiment, samples from normal tissues can be compared against those of the cancerous tissue to identify differences in the DNA sequence of those tissues. Thus, through the massively parallel sequencing provided by the 454 sequences, and similar instruments, 100,000 or more DNA samples can be parallel amplified. These sequences can then be compared against each other to look for genetic differences.

Computer programs exist to process this genetic information and locate the genetic discrepancies. Using current sequencers, it is possible to compile DNA sequence data into databases or other software such that sequences can be analyzed quickly and provide useable data within a very short period of time for potentially thousands of samples.

In one embodiment of this invention, hundreds or even thousands of microsatellite repeat regions can be amplified and compared across a series of a patient's normal vs. cancerous tissues. In another embodiment, hundreds or even thousands of microsatellite repeat regions can be amplified and compared across a series of a patient's cancerous tissues vs. cancerous tissues.

In another embodiment, hundreds or even thousands of microsatellite repeat regions can be amplified and compared across a series of multiple patients' normal vs. cancerous tissues. In another embodiment, hundreds or even thousands of microsatellite repeat regions can be amplified and compared across a series of multiple patients' cancerous tissues vs. cancerous tissues. Such applications could allow for multi-dimensional analyses of normal vs. tumor tissues and tumor vs. tumor tissues across individuals, families, and even general populations.

In another embodiment, hundreds or even thousands of microsatellite repeat regions can be amplified and compared to noncancerous, diseased tissues. A practical application of such an embodiment could include the analysis of microsatellite repeat regions and/or regions of interest within families with inherited cancer or other diseases.

In another embodiment of the present invention, the DNA from a cancerous tissue from a single patient can be compared against the same, but normal, tissues of thousands or even tens of thousands of non-cancerous individuals. In this embodiment, the DNA is generally isolated from the same tissue. Then, using the distinct tagging and massively parallel sequencing of the present invention, one can quickly yields massive amounts of genetic information that can be filtered and processed using standard computer programs.

6. Method for Determining Choice of Treatment in Patients

A method for determining choice of treatment in a patient previously diagnosed with cancer and other diseases involving altered DNA repair comprising: (a) ligating a universal linker to one or more DNA fragments, (b) denaturing the one or more DNA fragments, (c) hybridizing the one or more DNA fragments with a tagged oligonucleotide, wherein the tagged oligonucleotide is complementary to the region of interest, (d) capturing the tagged oligonucleotide, (e) recovering the one or more DNA fragments containing the region of interest, (f) making the one or more DNA fragments double-stranded, (g) optionally removing the universal linker, and (h) processing the one or more DNA fragments through parallel sequencing to aid in determining choice of treatment in a patient previously diagnosed with cancer and other diseases involving altered DNA repair. In one embodiment, steps (c)-(f) of the method are repeated before proceeding to step (g).

This embodiment aids in determining proper treatment regimens of diseased states in patients based on alterations within the genome of the patient. A more comprehensive analysis of one's DNA makeup, and possible mutations therein, could provide doctors or scientists with information that could be useful in making clinical decisions. During the course of treatment for cancer or other diseases, diseased states or the likelihood of future diseased states, caused by other genetic mutations may be identified. Based on such information, the doctor and patient can determine the best choice of treatment for such a patient. With the potential depth and breadth of coverage using the described invention, doctors could potentially screen thousands of genetic markers or loci in a relative short period of time with increased ease over current technologies.

### Methodology:

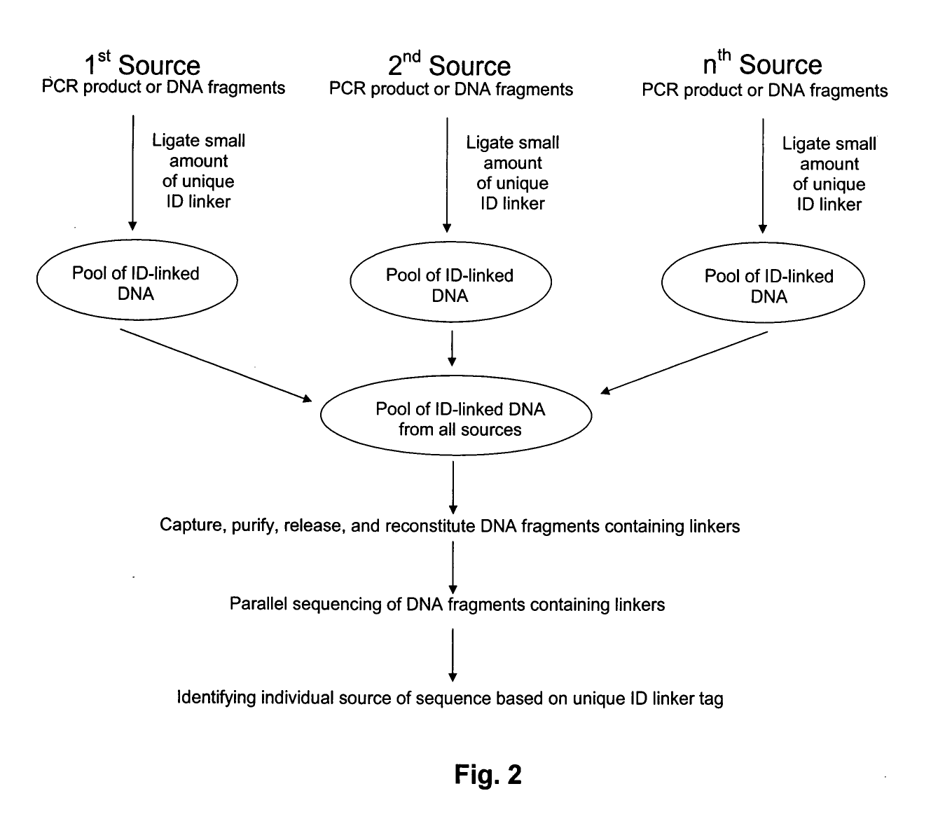

The general approach in carrying out the various embodiments of this invention is illustrated in FIG. 2. It is understood that FIG. 2 is only a general approach and that the various embodiments of this invention can be achieved in many different ways as exemplified herein and as the invention makes known to the person of ordinary skill in this field. In one embodiment, DNA fragments of interest are amplified, or otherwise isolated, from multiple individuals. The fragment, or amplicon, concentrations are normalized (e.g. via quantification and dilution). Fragments, or amplicons, from the same individual are pooled. Next, a linker containing a unique identification linker tag (ID) is ligated to each individual's fragments, or amplicons. Next, all individuals' fragments, or amplicons, are pooled together into a single pool and processed via emPCR, etc., as normal for parallel sequencing. Following sequencing, each unique identification linker tag is used to determine which sequence is associated with each source (cf. Binladen et al., 2007).

In one embodiment, DNA fragments of interest are amplified, or otherwise isolated, from multiple individuals. A small amount of a unique identification linker tag is ligated to each individual's DNA fragments, or amplicons. As used herein, a “small amount” is an amount such that there could be less probability of obtaining two tags on the same DNA fragment than having a single tag. This is accomplished by having an excess of DNA fragments present. The ratio is generally less than a 1:1 ratio of linker to DNA fragment. In other embodiments, this excess could be 10 fold excess of DNA fragments. In other embodiments, this excess could be more than 1000 fold excess of DNA fragments. The DNA fragments, or amplicons, from the same individual are pooled, followed by pooling of all individuals into a single pool. The DNA fragments, or amplicons, with linkers are captured and processed via emPCR, etc., as normal for parallel sequencing. Following sequencing, use the individual unique identification linker tags to determine which sequence is associated with each individual or source (cf. Binladen et al., 2007).

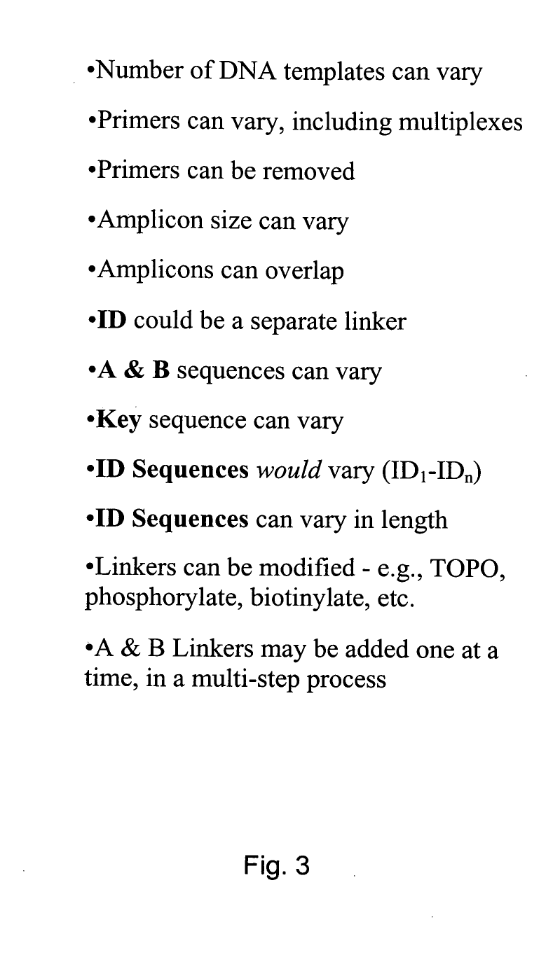

A large number of variations on these basic approaches are functionally equivalent. Many such variations are noted in FIG. 3. Additional variations and embodiments are possible, as explained below, and as would be apparent to those of skill in the art.

In one embodiment, normalization can be achieved by adding a very small number of modified (e.g., biotinylated) unique identification linker tags (ID) to individual PCR products, ligating, and then pooling across loci and individuals. Further modification (e.g., addition of TOPO isomerase) to the linkers could further increase the efficiency of this approach. In another embodiment, primers may be removed following PCR and before ligating on the identification linker tag (ID). This will reduce the amount of non-informative sequence substantially. For this: a) design primers that yield amplicons with restriction sites near the primer binding sites (the restriction site may even be within the primer, ideally near the 3′ end)—either one enzyme with two sites could be used or two different enzymes and recognition sites could be used; b) the recognition site(s) for the restriction enzyme(s) should be absent from the amplicon, except near/within the primers; c) after PCR, digest the amplicon with appropriate restriction enzyme(s); and d) eliminate the digested portion of the primers (e.g., via PEG precipitation).

In one embodiment, the unique identification linkers could be added separately from the 454 sequencing enabling linkers. This would increase the number of steps necessary for the method, but would give greater flexibility.

In one embodiment, unique identification linker tags can vary in length. A non-limiting example would include an 8-mer=4⁸ possible tags=65,536. Thus, with an 8-mer, it is possible to differentiate over approximately 60,000 different patient samples. With a 9-mer, 260,000 different patient samples can be easily differentiated.

In other embodiments, two different unique linker tags are attached to a single DNA molecule. Under this embodiment, it is possible to use a combination of shorter linker tags and still increase the number of DNA samples analyzed. This is possible because linker tags with two different unique tags generate more combinations of possible nucleotide sequences that could be used for a tag of a specific length (number of nucleotides) compared to a single unique linker tag of the same specific length.

In one embodiment, only one unique identification linker tag can be ligated to both or only one end of the DNA fragments. If fragment length is ≦read length or if fragments are generated from physical shearing, then use of only one unique linker tag is sufficient. In another embodiment, both ends are tagged with the same linker to sequence non-overlapping, or not completely overlapping, information from each end (i.e., fragments >read length). In another embodiment, one end is tagged with one unique identification linker tag and the other end is tagged with a different unique identification linker tag.

This invention allows researchers to efficiently conduct research that requires DNA sequencing in 3-dimensional parameter space—Number of Individuals, Number of Loci, and Depth of Coverage. Current approaches for 454 DNA sequencing limit parameter space to 2 dimensions—Depth of coverage and either Number of Individuals or Number of Loci [2nd dimension limited to the number of physical gaskets that fit on the plate (<16)]. Binladen et al., (2007) show that 2-dimensional parameter space can be expanded, but they are not explicit about this and have a generally poor solution to the problem. By taking advantage of all three dimensions, the number of potential studies that could benefit from 454 technology increases astronomically.

A non-limiting list of applications for this invention include: (1) mapping genomes (e.g., an entire vertebrate genome could be mapped in one or a few 454 runs), (2) population genetic surveys (e.g., determining sequence information for 32 individuals×120 species×5 mtDNA regions of ˜800 bp each [at an average coverage of 10×] in a single 454 run), (3) comparative genomics (e.g., determining sequence information for 96 individuals×400 DNA fragments/loci at an average coverage of 10× in a single 454 run), and (4) batch processing of multiple DNA samples on any parallel sequencing run (e.g., sequencing multiple bacterial genomes, pooling DNA templates of interest from multiple researchers, etc.). Because a very large number of unique ID's are possible, it would be possible for large numbers of researchers to provide samples to a sequencing center, each could be uniquely tagged, and then large numbers of samples pooled and run at relatively low cost.

The final product should be normalized for markers and individuals. A non-limiting example is shown in EXAMPLE 1.

One needs to be able to reliably capture and sequence the same approximately <1% of a typical vertebrate genome. Ideally this DNA would have a mutation rate higher than the average single-copy DNA in the genome so that the assay is efficient, yet yields results that are applicable to the entire genome. By focusing on the appropriate subset of DNA which serves as a sentinel for the genome, direct comparisons within and among samples can be made.

Microsatellite loci represent a few percent of the genome of humans, medaka, and most other eukaryotes. Tandem repeats are now known to be reliable sentinels for many different types of DNA within the genome (e.g., Barber et al., 2006; Singer et al., 2006). By targeting microsatellite DNA loci, the desired proportion of the genome can be captured using the enrichment techniques of Glenn & Salable (2005). Microsatellites are well known to have mutation rates that average about 1×10³ per locus per generation (e.g., Ellegren, 2000), which is many thousands of times higher than average single-copy nuclear DNA (cf. Haag-Liautard et al., 2007). In addition to the microsatellites themselves, the flanking DNA near microsatellites is also known to have elevated mutation rates relative to the rest of the genome (Glenn et al., 1996). Thus, the assay can target not only short repeats that can be sequenced entirely, but also just the DNA flanking repeats (i.e., it is not necessary to sequence across the entire repeat to determine repeat number).

By targeting a subset of microsatellite sequences (e.g., only DNA with or near (AGG)ₙ repeats), an even smaller portion of the genome can be assayed. If a sufficiently small portion of the genome is targeted (e.g., 0.01%) then DNA from multiple individuals can be tagged uniquely and combined into one assay, such that multiple comparisons can be done in one run of the 454 (e.g., 100 individual samples each with a unique tag can be assayed at one time for the same set of loci). Comparisons can then be made among cells: within organs or tissues (e.g., tumor vs. normal tissue or normal vs. diseased tissue within an individual), among organs or tissues (e.g., an organ that is the target of a drug or where a drug is detoxified vs. organs with little drug interaction; i.e., brain vs. liver vs. muscle, etc.), among individuals (e.g., parents and offspring or any pedigree), among populations (e.g., individuals or pedigrees in more vs. less polluted areas) and among species (e.g., response in humans, model organisms, and novel models).

Small insert DNA libraries enriched for microsatellite DNA loci can be produced and used in the present invention. It is straightforward to make the libraries with the desired qualities (Glenn and Schable, 2005; http://www.uga.edu/srel/DNA_Lab/protocols.htm). The methods of the present invention have validated samples from medaka and mice which are known to have higher mutation rates of STRs and ESTRs (respectively) within families than the relevant controls.

Although exemplified as a way to test for germ-line mutations (i.e., within pedigrees; e.g., between parents exposed to mutagens and their unexposed offspring), one embodiment of the current invention is applicable to assays of mutation rates among cells or cell populations within individuals. Thus, one could develop diagnostic assays indicative of DNA repair alteration which would suggest specific treatment options for various types of cancer and other disorders involving DNA repair.

Although it would be good to have a full genome sequence of the organisms being assayed, it is not required. This technique would be an efficient way to obtain the data needed to map the genome of an organism.

In one embodiment of the present invention, it would also be possible to compare the genomes of individual cells (e.g., individual sperm) by isolating the individual cells (e.g., by cell sorting), isolating the DNA, (optionally doing whole genome amplification), doing a restriction digest and linker ligation, capturing the targets, (optional amplification at this point), and assaying the enriched DNA.

The enrichments do not have to be microsatellite DNA loci. In some embodiments, any single, or mixture, of molecules (e.g., oligonucleotides, nucleic acids, and peptide nucleic acids) that can capture the desired proportion of the genome for analysis can produce the desired result. Microsatellite loci are, however, especially desirable for mutation assays because the mutation rate for the number of repeats and their flanking DNA is elevated relative to the rest of the genome.

It is also possible to compare the results obtained from the embodiments explained above to results from transgenes (e.g., in the Lambda medaka of R. Winn, UGA), and from STRs or ESTRs. Further applications include the use in studying naturally occurring and induced parthenogens, or clonally produced (100% homozygous) transgenic parents, to look at all the desired measures at one time; generating medaka with these characteristics, to expose them to model mutagens, and to assay them at the transgene; and exposing medaka to model mutagens under exposure scenarios we have better characterized for STRs.

### REFERENCES

Albert et al. (2007). Direct selection of human genomic loci by microarray hybridization. Nature Methods 4:903-905.
Barber et al. (2006). Radiation-induced transgenerational alterations in genome stability and DNA damage. Oncogene 25:7336-7342.
Binladen et al. (2007). The use of coded PCR primers enables high-throughput sequencing of multiple homolog amplification products by 454 parallel sequencing. PLoS ONE 2(2): e 197.doi:10.1371/journal.pone.0000197.
Dubrova (2003). Radiation-induced transgenerational instability. Oncogene 22, 7087-7093.
Ellegren (2000). Microsatellite mutations in the germline: implications for evolutionary inference. Trends in Genetics 16, 551-558.
Glenn and Schable (2005). Isolating microsatellite DNA loci. Methods in Enzymology 395:202-222.
Glenn et al. (1996). Allelic diversity in alligator microsatellite loci is negatively correlated with GC content of flanking sequences and evolutionary conservation of PCR amplifiability. Molecular Biology and Evolution 13:1151-1154.
Haag-Liautard et al. (2007). Direct estimation of per nucleotide and genomic deleterious mutation rates in Drosophila. Nature 445:82-85.
Russell (1951). X-ray-induced mutations in mice. Cold Spring Harbor Symposia on Quantitative Biology, 16: 327-336.
Russell et al. (1979). Specific-locus test shows ethylnitrosourea to be the most potent mutagen in the mouse. Proceedings of the National Academy of Sciences, USA, 76:5818-5819.
Shima and Shimada (1994). The Japanese Medaka, Oryzias latipes, as a new model organism for studying environmental germ-cell mutagenesis. Environmental Health Perspectives 102(Suppl 12):33-35.
Singer et al. (2006). Detection of induced male germline mutation: Correlations and comparisons between traditional germline mutation assays, transgenic rodent assays, and expanded simple tandem repeat instability assays. Mutation Research doi: 10.1016/j.mrfmmm.2006.01.017.
Yank (2004). Advances in the application of germline tandem repeat instability for in situ monitoring. Mutation Research-Reviews in Mutation Research 566, 169-182.
   The above references are herein incorporated by reference in their entirety.

### EXAMPLES

The following examples are included to demonstrate certain embodiments of the invention. It should be appreciated by those of skill in the art that the techniques disclosed in the examples represent techniques that function well in the practice of the invention. However, those of skill in the art should, in light of the present disclosure, appreciate that many changes can be made in the specific embodiments which are disclosed and still obtain a similar result without departing from the scope of the invention.

### Example 1

The product should be normalized for markers and individuals. A non-limiting example is described below.

First, primers are designed to ensure that no/few primer dimers form and a product of ˜400 bp (if desired, ensure restriction sites are close, or internal, to primers). Using the designed primers, PCR a single marker for 96 (or any number of) individuals. Run out a few microliters of the PCR products for a single row or column to ensure the PCRs generally worked.

Optionally, to eliminate primers and reduce wasted sequence, cut the PCR products with restriction enzyme(s), blunt the ends if restriction enzyme(s) do not leave blunt ends, and remove external portions of PCR (anything less than ˜130 bp) via PEG precipitation. If a TOPO linker tag is used at the next step, the addition of an incubation with Taq and dATP or tdt and ddATP (and appropriate buffers, etc.) is required.

Next, add a very small amount (e.g., 1000 or 100,000 molecules) of a biotin_{—}454-A_ID_TOPO Linker (SEQ IDS:1, 2, 7, or 8) or biotin_{—}454-A_ID Linker (SEQ IDS:23-24). to each PCR (this will randomly grab 1000 or 100,000 molecules by one end or the other; nearly no chance for both ends to get a linker).

Repeat the preceding steps above for multiple markers.

Next, pool the markers and the individuals. Once pooling is done, capture the tagged DNA on streptavidin paramagnetic beads (or any system desired to capture the biotin). Wash the mixture to get rid of non-captured DNA. Once DNA is captured on the beads and washed, progress to one of four options below (dependent on the application)

Option 1:

Phosphorylate the DNA (on the beads) with T4 PNK (or any other acceptable polynucleotide kinase) and wash again. Next, elute (via heat) the strand complementary to the biotinylated strand. Make double-stranded with the 454-A primer (Integrated DNA Technologies, Inc.). Add the 454-B_linker (Invitrogen) (not phosphorylated, but also with biotin on the 5′ end). Finally, treat as normally done for 454 libraries and emPCR.

Option 2:

Elute (via heat) the strand complementary to the biotinylated strand and phosphorylate ssDNA with PNK. Heat to 95° C. Make double-stranded with the 454-A primer. Add the 454-B_linker (not phosphorylated, but also with biotin on 5′ end), and treat as normally done for 454 libraries and emPCR.

Option 3 (if Labile Biotin is Used):

Release desired fragments as dsDNA and process library for 454 sequencing as normal (sequencing from one end only).

Option 4 (if Labile Biotin is Used): Release desired fragments as dsDNA and add the 454-B_linker (not phosphorylated, but also with biotin on 5′ end). Treat as normally done for 454 libraries and emPCR.

### Example 2

Several experiments to demonstrate proof of concept for the Identification (ID) Linkers, Topo-Normalization, and capture of relevant products for mutation detection are described below. Although it is equally easy to configure Topoisomerase charged linkers with blunt ends (such as would be used with randomly sheared and end repaired DNA or PCR products generated from DNA polymerases without non-template addition of adenine), we focused on linkers that could be used with PCR products generated with Taq DNA polymerase to demonstrate the proof of concept.

Starting Materials:

Three separate oligos were ordered for each linker and are displayed here as assembled double-stranded product ready for charging with Topoisomerase. (SEQ ID NOS: 1-6) (Invitrogen). The charged Topo-linkers were then obtained.

5′Biotin 454A ID1 TOPO-TA

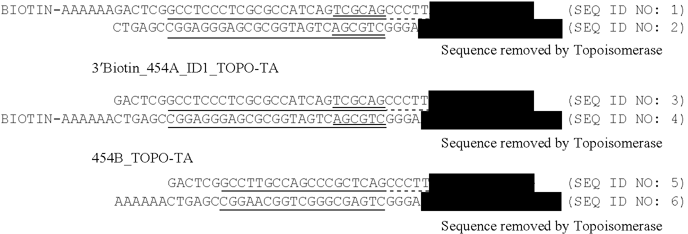

The experiments below focus on the following linkers (SEQ ID NOs: 7-10):

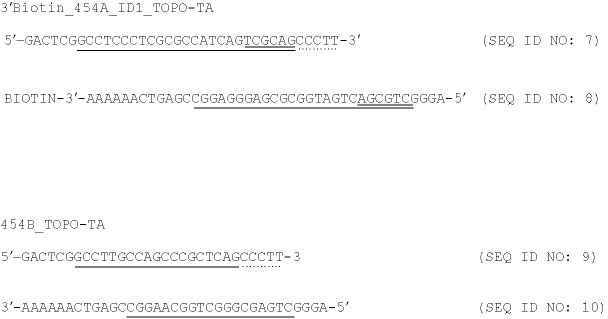

Experiments progressed along two general themes: 1) cloning and sequencing, or 2) quantitative PCR.

Cloning and Sequencing.

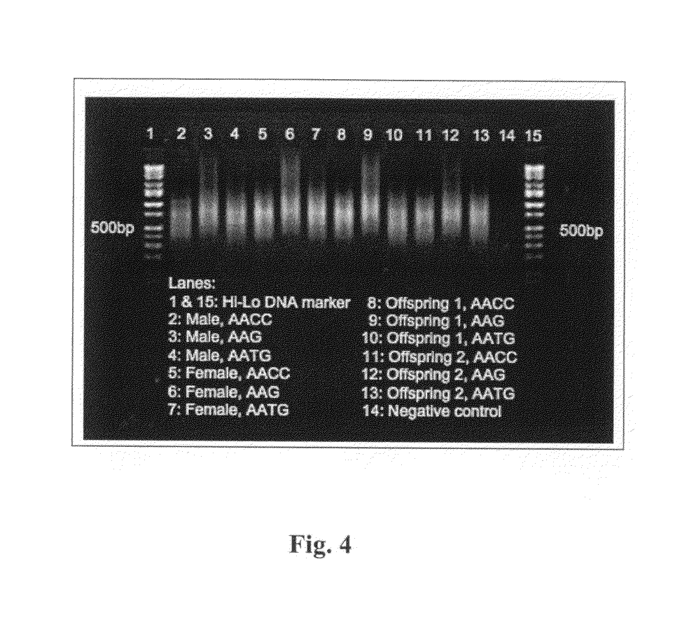

Samples appropriate for mutation detection were prepared using microsatellite oligonucleotide repeat capture. Four Mus samples were enriched (one irradiated male, the female mate, and two of their offspring) for simple tandem repeats with three unique core sequences: AAG, AATG, and AACC. A protocol described in Glenn & Schable, 2005, that has been used to develop STR enriched libraries for more than 100 eukaryotic species, was followed. In brief, DNA was digested with the restriction enzyme Rsa I (New England Biolabs) and simultaneously ligated to a custom linker (SuperSNX, see Glenn & Schable, 2005). DNA was then denatured and hybridized to one of three biotinylated oligonucleotides: (AAG)₈, (AACC)₅, or (AATG)₆. The biotinylated DNA was captured on streptavidin coated paramagnetic beads and unhybridized DNA was washed away. Hybridized DNA was eluted and amplified by PCR. The smears illustrate that STRs and their flanking sequence for a range of sizes were captured. (FIG. 4). These four samples that are enriched for the three STRs are thus ready for linker ligation in preparation for 454 sequencing.

Those samples and others prepared using the methods of Glenn & Schable (2005), were modified by adding 3′Biotin_{—}454A_ID1_TOPO-TA (SEQ ID NOS: 3-4), followed by 454B_TOPO-TA (SEQ ID NOS 9-10). The products were amplified using the 454_A and 454_B primers using PCR. The resulting PCR amplicons were then cloned with TOPO-TA Cloning Kits (Invitrogen). Inserts from clones were isolated using bacterial colony PCR with M13 forward and reverse primers and sequenced using the same primers and BigDye v3.1 (Applied Biosystems) on an ABI-3130x1 sequencer. Sequences from both strands were assembled and edited in Sequencher 4.5 (Genecodes). Incorporation of the linkers was achieved, as demonstrated by the following three examples. The expected sequence (SEQ ID NOS: 12, 14, 16, 18, 20, 22) is given below the actual sequence (SEQ ID NOS: 11, 13, 15, 17, 19, 21). The 454_A_LINKER sequence is underlined ( [symbol] ). The 454_B_LINKER is denoted by ( [symbol] ). The ID sequence is double underlined ( [symbol] ). The TOPO cloning site is denoted by ( [symbol] ). Deletions of base pairs in the actual sequence are grey highlighted on the expected sequence.

1) Pelican_{—}24 has linkers on both ends, but has a single bp deletion

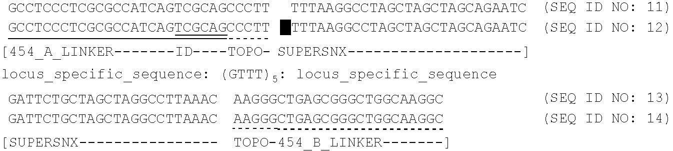

2) Mus_1674_11 has both linkers, but has a 5 bp deletion in superSNX on the A-Linker side:

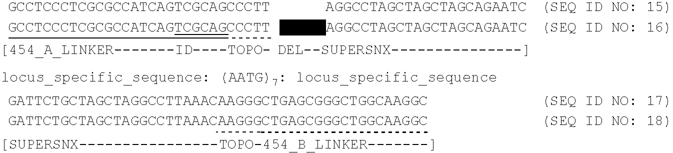

3) Mus_2148_8 has both linkers, but has a 13 bp deletion in superSNX on the B-Linker side:

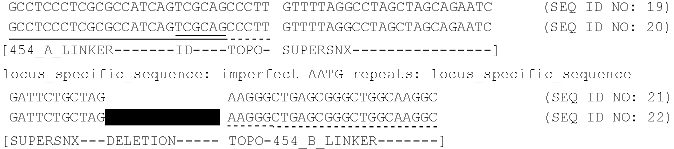

Thus, these experiments demonstrate: 1) the Topo-linker strategy works to quickly introduce 454A an 454B sequences needed, as well as identification (ID) sequences incorporated into the tags, and 2) that sequences with microsatellites can be captured and manipulated allowing mutation rate assessment if sequenced at sufficient depth. An unexpected bonus is that some of the primer-sequence is deleted, thus reducing unnecessary sequence obtained from this approach.

Quantitative PCR Results.

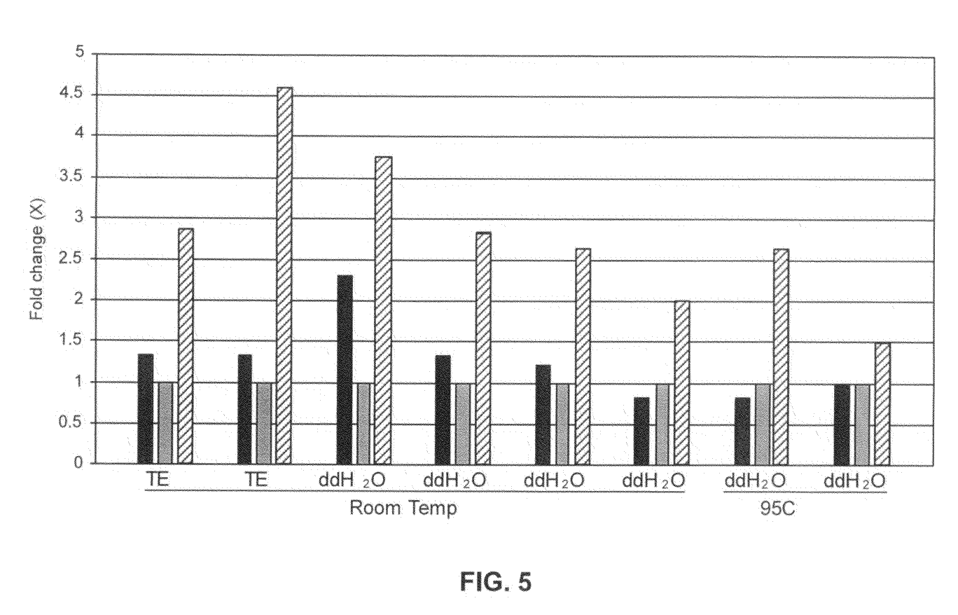

Three different quantities of PCR product were used to test for normalization: 4.5 μg of PCR1 (FIG. 5, hashed bars), 1.5 μg PCR2 (FIG. 5, dark shaded bars), 0.25 μg of PCR3 (FIG. 5, light shaded bars) each with 1 μl 3′Biotin_{—}454A_ID1_TOPO-TA-linker (SEQ IDS:7-8) in individual tubes. These were incubated at 37° C. for 20 minutes. Next, an over-abundance of 454B was added to each. The samples were then nick repaired using 1×PCR mastermix with no primer. The three nick-repaired products were then pooled with the beads and captured in 1×hyb buffer (6×SSC, 0.1% SDS). The beads were washed twice with 2× wash buffer (2×SSC, 0.1% SDS), twice with 1× wash buffer (1×SSC, 0.1% SDS) (20×SSC=3.0 M NaCl, 0.3 M sodium citrate, pH 7.0), twice with TE (10 mM Tris pH 8; 0.2 mM EDTA pH 8) (kept elutant), four times in ddH2O (kept elutant). Next, they were eluted in ddH2O at 95° C. (kept elutant). Finally, they were eluted again in ddH2O at 95° C. (kept).

The amount of DNA was then quantified by quantitative PCR (qPCR) using 454A and 454B primers. Normalization improves with more washes (FIG. 5). Initially, the amount of PCR products varied about 20-fold. The first heated elution reduced the variance to about 4× with a lot of product released. The second heat was normalized to within the measurement error of qPCR, but yielded far less DNA.

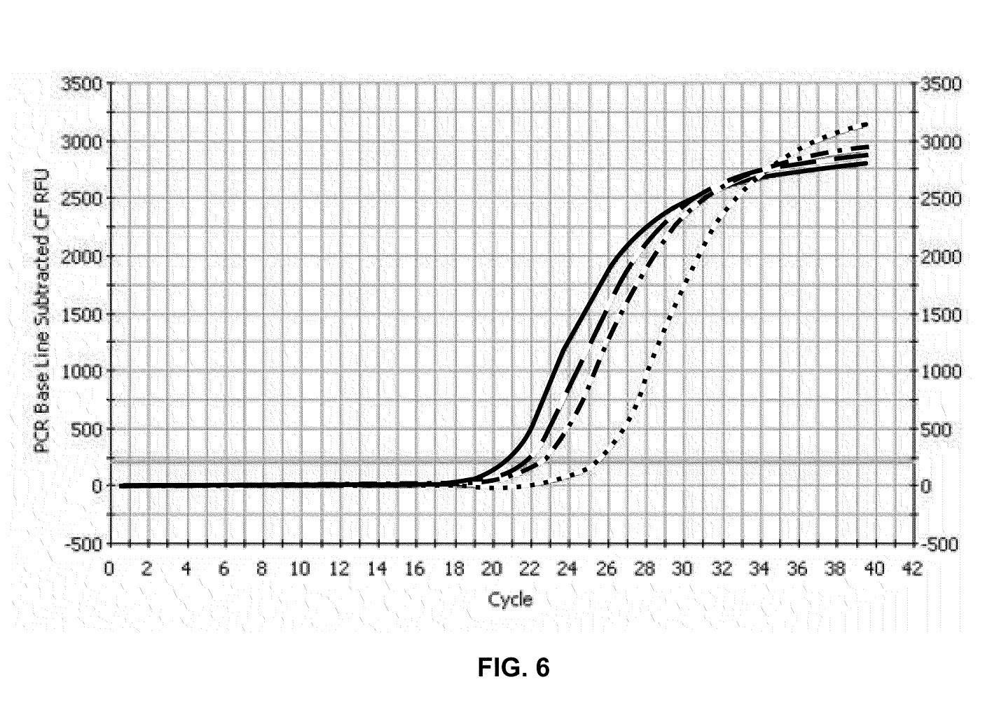

An additional experiment was done following similar methods to those used above. However, this time a single PCR product diluted to varying amounts (1000 ng, 100 ng, 10 ng, and 1 ng; (*, +, o, x respectively on FIG. 6)) was used. The amount of product, as judged by qPCR, was similar for the first three (FIG. 6), showing that 100-fold variance in PCR product can be reduced to <4-fold variation.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

The claims in the instant application are different than those of the parent application or other related applications. The Applicant therefore rescinds any disclaimer of claim scope made in the parent application or any predecessor application in relation to the instant application. The Examiner is therefore advised that any such previous disclaimer and the cited references that it was made to avoid, may need to be revisited. Further, the Examiner is also reminded that any disclaimer made in the instant application should not be read into or against the parent application.

1. A method to tag one or more DNA fragments from one or more subjects to investigate one or more loci of interest comprising:
(a) normalizing the concentration of the one or more DNA fragments;
(b) pooling the one or more DNA fragments;
(c) ligating distinct identification linker tags to each of the DNA fragments;
(d) optionally pooling the distinctly tagged DNA fragments;
(e) processing the distinctly tagged DNA fragments through parallel sequencing; and
(f) using the identification linker tag to differentiate the one or more DNA fragments to investigate one or more loci of interest.

2. The method of claim 1, wherein the one or more DNA fragments comprise DNA fragments from different sources.

3. The method of claim 2, wherein the different sources comprise different tissue types.

4. The method of claim 2, wherein the different sources comprise different subjects.

5. The method of claim 1, wherein the distinctly tagged DNA fragments of step (d) are pooled from a plurality of different sources.

6. The method of claim 4, wherein each source has a distinct identification linker tag.

7. The method of claim 1, wherein one or more additional linkers are ligated to the DNA fragment prior to parallel sequencing.

8. The method of claim 5, wherein the one or more additional linkers contain unique DNA sequences.

9. The method of claim 1, wherein the identification linker tag is a 1 mer to about a 100 mer.

10. The method of claim 1, wherein only one end of the one or more DNA fragments is tagged with the identification linker tag.

11. The method of claim 1, wherein both ends of the one or more DNA fragments are tagged with the same identification linker tag.

12. The method of claim 1, wherein the identification linker tag comprises biotin, digoxigenin, or FITC.

13. The method of claim 1, wherein the identification linker tag comprises topoisomerase.

14. The method of claim 1, wherein the identification linker tags are ligated individually, simultaneously, or in multiple steps.

15. The method of claim 1, wherein the one or more DNA fragments are isolated or amplified from genomic DNA, plasmid DNA, or mitochondrial DNA.

16. The method of claim 1, wherein the identification linker tag is captured for sequencing through hybridization to oligonucleotides attached to a solid support.

17. A method to efficiently tag and/or normalize one or more DNA fragments comprising:
(a) ligating a predefined amount of an identification linker tag to one or more DNA fragments;
(b) pooling the one or more tagged DNA fragments;
(c) repeating steps (a)-(b) for each subject;
(d) optionally pooling the one or more tagged DNA fragments for all subjects;
(e) capturing the one or more tagged DNA fragments;
(f) purifying the one or more tagged DNA fragments;
(g) releasing and reconstituting the one or more tagged DNA fragments;
(h) processing the one or more tagged DNA fragments through parallel sequencing; and
(i) using the identification linker tag to differentiate the one or more tagged DNA fragments from one or more subjects.

18. The method of claim 17, wherein one or more additional linkers are ligated to the DNA fragment prior to parallel sequencing.

19. The method of claim 17, wherein the one or more additional linkers contain unique DNA sequences.

20-22. (canceled)

23. The method of claim 17, wherein step 17(d) is omitted and/or both ends of the one or more DNA fragments are ligated to linkers lacking unique identification sequences.

24. (canceled)

25. The method of claim 17, wherein the identification linker tag is captured for sequencing through hybridization to oligonucleotides attached to a solid support.

26. The method of claim 17, wherein the identification linker tag is phosphorylated.

27. The method of claim 17, wherein the identification linker tag comprises topoisomerase.

28-29. (canceled)

30. The method of claim 17, wherein the one or more DNA fragments are isolated or amplified from genomic DNA, plasmid DNA, or mitochondrial DNA.

31. A method for the measurement of mutations in the genome of a cell population by identifying and sequencing a region of interest comprising:
(a) ligating a universal linker to one or more DNA fragments;
(b) denaturing the one or more DNA fragments;
(c) hybridizing the one or more DNA fragments with a tagged oligonucleotide, wherein the tagged oligonucleotide is complementary to the region of interest;
(d) capturing the tagged oligonucleotide;
(e) recovering the one or more DNA fragments containing the region of interest;
(f) making the one or more DNA fragments double-stranded;
(g) optionally removing the universal linker; and
(h) processing the one or more DNA fragments through parallel sequencing to aid in the direct measurement of mutations in the genome of the cell population.

32. The method of claim 31, wherein steps (c)-(f) are repeated before proceeding to step (g).

33. The method of claim 31, wherein the DNA is obtained by whole genome amplification.

34. The method of claim 31, wherein the tagged oligonucleotide is captured for sequencing through hybridization to oligonucleotides attached to a solid support.

35. The method of claim 31, wherein the region of interest contains a region or regions with at least one microsatellite repeat.

36. The method of claim 31, wherein the universal linker comprises an identification linker tag.

37. The method of claim 31, wherein a peptide nucleic acid is used at step (c).

38. The method of claim 31, wherein the universal linker is not removed.

39. A method of comparing a portion of the genomes of one or more cells comprising:
(a) ligating a universal linker to one or more DNA fragments;
(b) denaturing the one or more DNA fragments;
(c) hybridizing the one or more DNA fragments with tagged oligonucleotides, wherein the tagged oligonucleotides are complementary to the region of interest;
(d) capturing the tagged oligonucleotide;
(e) recovering the one or more DNA fragments containing the region of interest;
(f) making the one or more DNA fragments double-stranded;
(g) optionally removing the universal linker; and
(h) processing the one or more DNA fragments through parallel sequencing to aid in comparing the genomes of one or more cells.

40. The method of claim 39, wherein steps (c)-(f) are repeated before proceeding to step (g).

41-43. (canceled)

44. The method of claim 39, wherein the region of interest contains a region or regions with at least one microsatellite repeat.

45. The method of claim 39, wherein the region of interest is identified from DNA sequences conserved among divergent taxa.

46. The method of claim 39, wherein the oligonucleotides complementary to the region of interest include a repetitive element or flank repetitive elements.

47-49. (canceled)

50. The method of claim 39, wherein a peptide nucleic acid is used at step (c).

51. The method of claim 39, wherein the universal linker is not removed.

52. (canceled)

53. A method for diagnosing a disease comprising:
(a) ligating a universal linker to one or more DNA fragments;
(b) denaturing the one or more DNA fragments;
(c) hybridizing the one or more DNA fragments with a tagged oligonucleotide, wherein the tagged oligonucleotide is complementary to the region of interest;
(d) capturing the tagged oligonucleotide;
(e) recovering the one or more DNA fragments containing the region of interest;
(f) making the one or more DNA fragments double-stranded;
(g) optionally removing the universal linker;
(h) processing the one or more DNA fragments through parallel sequencing; and
(i) comparing the genomes of one or more cells to diagnose the disease in the patient.

54. The method of claim 53, wherein steps (c)-(f) are repeated before proceeding to step (g).

55-65. (canceled)

66. A method for determining choice of treatment in a patient previously diagnosed with cancer and other diseases comprising:
(a) ligating a universal linker to one or more DNA fragments;
(b) denaturing the one or more DNA fragments;
(c) hybridizing the one or more DNA fragments with a tagged oligonucleotide, wherein the tagged oligonucleotide is complementary to the region of interest;
(d) capturing the tagged oligonucleotide;
(e) recovering the one or more DNA fragments containing the region of interest;
(f) making the one or more DNA fragments double-stranded;
(g) optionally removing the universal linker; and
(h) processing the one or more DNA fragments through parallel sequencing to aid in determining choice of treatment in a patient previously diagnosed with cancer and other diseases involving altered DNA repair.

67. The method of claim 66, wherein steps (c)-(f) are repeated before proceeding to step (g).

68-78. (canceled)


SEQUENCE LISTING (24 sequences)

SEQ ID NO: 1 - DNA, 54 bp, Artificial Sequence
  Synthesized 5′Biotin_454A_ID1_TOPO-TA
aaaaaagactcggcctccctcgcgccatcagtcgcagcccttattccgatagtg

SEQ ID NO: 2 - DNA, 51 bp, Artificial Sequence
  Synthesized 5′Biotin_454A_ID1_TOPO-TA
ctgagccggagggagcgcggtagtcagcgtcgggaataaggctatcacaac

SEQ ID NO: 3 - DNA, 48 bp, Artificial Sequence
  Synthesized 3′Biotin_454A_ID1_TOPO-TA
gactcggcctccctcgcgccatcagtcgcagcccttattccgatagtg

SEQ ID NO: 4 - DNA, 57 bp, Artificial Sequence
  Synthesized 3′Biotin_454A_ID1_TOPO-TA
aaaaaactgagccggagggagcgcggtagtcagcgtcgggaataaggctatcacaac

SEQ ID NO: 5 - DNA, 42 bp, Artificial Sequence
  Synthesized 454B_TOPO-TA
gactcggccttgccagcccgctcagcccttattccgatagtg

SEQ ID NO: 6 - DNA, 51 bp, Artificial Sequence
  Synthesized 454B_TOPO-TA
aaaaaactgagccggaacggtcgggcgagtcgggaataaggctatcacaac

SEQ ID NO: 7 - DNA, 36 bp, Artificial Sequence
  Synthesized 3′Biotin_454A_ID1_TOPO-TA
gactcggcctccctcgcgccatcagtcgcagccctt

SEQ ID NO: 8 - DNA, 41 bp, Artificial Sequence
  Synthesized 3′Biotin_454A_ID1_TOPO-TA
aaaaaactgagccggagggagcgcggtagtcagcgtcggga

SEQ ID NO: 9 - DNA, 30 bp, Artificial Sequence
  Synthesized 454B_TOPO_TA
gactcggccttgccagcccgctcagccctt

SEQ ID NO: 10 - DNA, 35 bp, Artificial Sequence
  Synthesized 454B_TOPO_TA
aaaaaactgagccggaacggtcgggcgagtcggga

SEQ ID NO: 11 - DNA, 53 bp, Artificial Sequence
  Actual Pelican_24 linker
gcctccctcgcgccatcagtcgcagccctttttaaggcctagctagcagaatc

SEQ ID NO: 12 - DNA, 54 bp, Artificial Sequence
  Expected Pelican_24 linker
gcctccctcgcgccatcagtcgcagcccttgtttaaggcctagctagcagaatc

SEQ ID NO: 13 - DNA, 48 bp, Artificial Sequence
  Actual Pelican_24 linker
gattctgctagctaggccttaaacaagggctgagcgggctggcaaggc

SEQ ID NO: 14 - DNA, 48 bp, Artificial Sequence; identical to SEQ ID NO: 13
  Expected Pelican_24 linker

SEQ ID NO: 15 - DNA, 49 bp, Artificial Sequence
  Actual Mus_1674_11 linker
gcctccctcgcgccatcagtcgcagcccttaggcctagctagcagaatc

SEQ ID NO: 16 - DNA, 54 bp, Artificial Sequence; identical to SEQ ID NO: 12
  Expected Mus_1674_11 linker

SEQ ID NO: 17 - DNA, 48 bp, Artificial Sequence; identical to SEQ ID NO: 13
  Actual Mus_1674_11 linker

SEQ ID NO: 18 - DNA, 48 bp, Artificial Sequence; identical to SEQ ID NO: 13
  Expected Mus_1674_11 linker

SEQ ID NO: 19 - DNA, 54 bp, Artificial Sequence
  Actual Mus_2148_8 linker
gcctccctcgcgacatcagtcgcagcccttgttttaggcctagctagcagaatc

SEQ ID NO: 20 - DNA, 54 bp, Artificial Sequence; identical to SEQ ID NO: 12
  Expected Mus_2148_8 linker

SEQ ID NO: 21 - DNA, 35 bp, Artificial Sequence
  Actual Mus_2148_8 linker
gattctgctagaagggctgagcgggctggcaaggc

SEQ ID NO: 22 - DNA, 48 bp, Artificial Sequence; identical to SEQ ID NO: 13
  Expected Mus_2148_8 linker

SEQ ID NO: 23 - DNA, 34 bp, Artificial Sequence
  Synthesized biotin_454-A_ID Linker
aaagactcggcctccctcgcgccatcagtcgcag

SEQ ID NO: 24 - DNA, 31 bp, Artificial Sequence
  Synthesized biotin_454-A_ID Linker
ctgagccggagggagcgcggtagtcagcgtc